(19) United States
(12) Patent Application Publication
(10) Pub. No.: US 2004/0077073 A1
(43) Pub. Date: Apr. 22, 2004

(54) Methods and apparatus for interactive micromanipulation of biological materials

(76) Inventors: Melvin Schindler, Okemos, MI (US); John F. Holland, Lansing, MI (US); Max R. Olinger, Holland, MI (US)

Correspondence Address: HARNESS, DICKEY & PIERCE, P.L.C., P.O. BOX 828, BLOOMFIELD HILLS, MI, 48303, US

(21) Appl. No.: 10/273,462

(22) Filed: Oct. 18, 2002

(51) Int. Cl. A61N 1/30; G01N 1/30; G01N 33/48; C12M 1/34

(52) U.S. Cl. 435/287.1; 604/20

(57) ABSTRACT

An apparatus for micromanipulating biological materials, comprising:

(a) a laser emitting light at a wavelength≧600 nm;

(b) a matrix supporting the biologic material, comprising a light-absorbing material; and

(c) a system for focusing light from the source onto specific regions of the matrix.

The light absorbing material absorbs the light and coverts it to heat so as to disrupt the matrix and the biological material at the point where the light contacts the matrix. Preferably, the matrix is supported by a carrier to form a bi-layer matrix composite. In another embodiment, the matrix is supported on a support plate having an aperture which is covered, at least in part, by the matrix. In another embodiment, the a matrix is supported by a carrier, wherein at least one of the matrix and the support plate comprises a cell growth modifier.

### BACKGROUND

The present invention relates to apparatus, materials, and methods for sorting, dissecting, ablating or otherwise manipulating materials on a microscopic level based upon differing characteristics. In particular, the present invention relates to the use of laser light to discriminate between populations of cells or other biological materials by selectively removing or destroying unwanted materials, the matrix supporting the material, or both.

A major focus in modern medical and pharmaceutical research relates to the modification of cell function by mediating genetic or other chemical processes within cells. Many experiments are done with isolated enzymes, receptors or other cell components. Unfortunately, observations using such isolated components may not accurately represent processes occurring in the living organism. Moreover, some processes may be detected only through observing cell function over a period of time, e.g., over several cycles of cell replication, thus requiring the cells to remain viable and observable throughout the experimental process.

A variety of methods and instruments have been developed for the purpose of analyzing individual cells and separating them into distinct sub-populations based on characteristics of interest. Such techniques are used in immunology, hematology, cell cycle analysis, pathology, biochemistry, cell biology, pharmacology, and toxicology. Diseases investigated with cell sorting technology include leukemia, lymphoma, and other cancers, infectious diseases, autoimmune diseases, and genetic disorders. These techniques have been applied to investigations of tumorgenesis and to the design of new anti-cancer drugs and therapies. One such technique, flow cytometry, is predominantly used in the characterization and separation of anchorage independent lymphoid cells. In flow cytometry, cells are sorted into separate tubes based on fluorescent or other light interactive behavioral differences. Sorted cells can be run through the system a number of times to provide further enrichment. The technique of laser micro-dissection has been used to obtain genetic material from micro-dissected fragments of tissue, so as to catalogue cellular heterogeneity within tumors. Similarly, recent efforts to isolate and manipulate single living cells from tissues and cell cultures have been shown to allow the selective cloning and proliferation of subpopulations of cells. This evidences a significant potential for the design of new clinical approaches that include autologous cellular therapies (ACTs) against tumors, and that can accomplish the isolation of stem/progenitor cells from tissues.

Mixed polymer substrates have been employed to separate cells based on surface properties, cell electrophoresis has been used to separate cells by surface charge, and solid phase lectins and immobilized antibodies have been exploited to differentiate between altered structural determinants in membrane proteins for subsequent cell separation. Measurements of luminescence, particularly fluorescence, can be used to discriminate among cell populations for the identification and separation of subpopulations of cells based on differences in bound luminescent probes (usually an antibody or lectin containing a covalently attached fluorophore).

In an effort to mimic the organization and specific growth patterns found between diverse cell populations found in tissues, a number of lithographic and other techniques have been used to create tissue culture surfaces that can promote the segregation and organization of diverse cell types into specific micro-patterns and shapes that mimic native tissue in form and function. Specific patterns of cell growth help to regulate, and are regulated by, altered patterns of gene expression. In this manner, functionally and topologically distinct domains of cells are formed within tissues for specific function. These research methods rely on the formation of regions on cell growth surfaces that are modified to facilitate or inhibit cell growth. In many instances, such modified surfaces have optimized the growth of multiple cell types in which two different populations of cells grow with specific geometric or proportional relationships to each other.

However, the utility of methods and instruments among those known in the art of cell and tissue processing and manipulation is limited, due to lack of availability of equipment (e.g., for photolithography and micro-machining), difficulty in avoiding contamination with unwanted cells, damaging cells of interest, the necessity in many systems to use suspended cells for separation, limitations in the quantity of cells that can be handled, and expense. Moreover, in some systems, cell differentiation is determined only by structural markers attached to the cells, rather than isolating and separating cells based on alterations in physiological activity.

### SUMMARY OF THE INVENTION

The present invention provides apparatus, materials and methods for interactive micromanipulation of cells, tissue, and other biological material. In particular, in one embodiment, this invention provides an apparatus comprising:

(a) a light source, preferably a laser, emitting light at a wavelength of at least about 600 nm (preferably red, near infra-red, or infra-red);

(b) a material matrix, comprising a light-absorbing material, wherein the light absorbing material selectively absorbs light at the wavelength of the light emitted by the light source; and

(c) a light direction system for focusing light from the light source onto specific regions of the matrix. In the operation of the apparatus, the light absorbing material absorbs the light and coverts it to heat so as to effect disruption of the matrix, and biological material on the matrix, at the point where the light contacts the matrix. Preferably, the light is infrared or near-infrared.

In a preferred embodiment, the matrix is adhered to, or otherwise supported by, a carrier to form a bi-layer matrix composite. In operation of the apparatus, focusing the light onto the matrix causes the matrix to be disrupted and adhere to the carrier at the point where the light is focused. In particular, the present invention also provides methods of micro-dissecting a biological material sample, comprising the steps of:

(a) placing the sample on a matrix which is adhered to, or otherwise supported by, a carrier;

(b) identifying a first area of the matrix upon which at least a portion of the sample has been placed, and a second area of the matrix contiguous to the first area;

(c) disrupting the matrix at the perimeter between the first and the second areas using a focused light beam, preferably generated by a laser; and

(d) separating the first area and the sample on the first area from the second area. Preferably the disrupting step is performed so as to weld or otherwise bond the matrix at the perimeter to the carrier. Also preferably, the first area remains adhered to the support plate, and the second area is removed from the support plate.

In another embodiment, the matrix is supported on a support plate having an aperture which is covered, at least in part, by the matrix. Associated methods comprise the steps of:

(a) placing a biological material sample on a matrix which is adhered to, or otherwise supported by, a support plate, wherein a region of the sample and associated matrix extends over an aperture in the support plate;

(b) identifying a first area of the matrix within said region, and a second area of the matrix contiguous to the first area;

(c) disrupting the matrix at the perimeter between the first and second areas using a focused light beam, preferably generated by a laser; and

(d) excising the first area and the sample on the first area by mechanical means applied to the side of the matrix opposite the side associated with the sample.

The present invention also provides a biological material platform comprising a matrix which is adhered to, or otherwise supported by, a carrier, wherein at least one of the matrix and the carrier comprises a cell growth modifier.

### BRIEF DESCRIPTION OF THE DRAWINGS

FIG. 1 is a schematic diagram of an apparatus embodiment useful in the methods of the present invention.

FIG. 2 is a side view of matrices useful in the methods of the present invention.

FIG. 3 depicts a biological material platform of the present invention and the steps of a method of this invention.

FIG. 4 depicts a biological material platform of the present invention, comprising a carrier having an aperture.

FIG. 5 depicts a cross-sectional view of biological material platforms of the present invention, comprising plates having an aperture and bi-layer matrix composites.

FIG. 6 is a cross-sectional view of a plate useful in the methods of this invention.

FIG. 7 depicts a biological material platform of the present invention, and the steps of a method of this invention.

FIG. 8 depicts the steps of a method of this invention for ablating unwanted biological material.

FIG. 9 depicts the steps of a method of this invention for cutting a substrate and ablating undesired biological material.

FIG. 10 depicts biological material platform according to the present invention.

FIG. 11 is a schematic diagram of the light direction system in a preferred interactive micromanipulation apparatus useful in the methods of this invention.

It should be noted that the figures set forth herein are intended to exemplify the general characteristics of an apparatus, materials and methods among those of this invention, for the purpose of the description of such embodiments herein. These figures may not precisely reflect the characteristics of any given embodiment, and are not necessarily intended to define or limit specific embodiments within the scope of this invention.

### DETAILED DESCRIPTION OF THE INVENTION

The present invention relates to apparatus, materials and methods for interactive micromanipulation of biological materials. As referred to herein, “biological materials” refers to any living material or material derived from living material, which may be viable, fixed or frozen, and component parts thereof, aggregates thereof, and combinations thereof. Biological materials include individual cells, cell components, and cell cultures of such origin as plants, bacteria, yeast, and humans or other animals. Biological materials also include tissues (e.g., organized, functionally differentiated, groups of cells) from humans, other animals, or plants. (As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention. Also as used herein, the word “include,” and its variants, is intended to be non-limiting, such that recitation of items in a list is not to the exclusion of other like items that may also be useful in the materials, compositions, devices, and methods of this invention.)

Preferably, the biological materials used in the methods of this invention are heterogeneous, wherein the materials, as a whole, comprise at least two component parts having dissimilar biological characteristics. Such biological characteristics include those relating to composition, physical properties, function, and behavior. As referred to herein, “micromanipulation,” or “interactive micromanipulation” (IMM), of biological materials is the manipulation of biological materials, or of their direction or pattern of growth or development. IMM includes methods of sorting, dissecting, or otherwise discriminating biological materials. In one embodiment, IMM comprises the separation of two or more biological materials or component parts thereof, based on one or more differing biological characteristics, at a microscopic level. IMM includes micro-dissection, single or multiple cell isolation, organization of cell growth into specific patterns, and producing specific proportions between different cell types.

### Apparatus

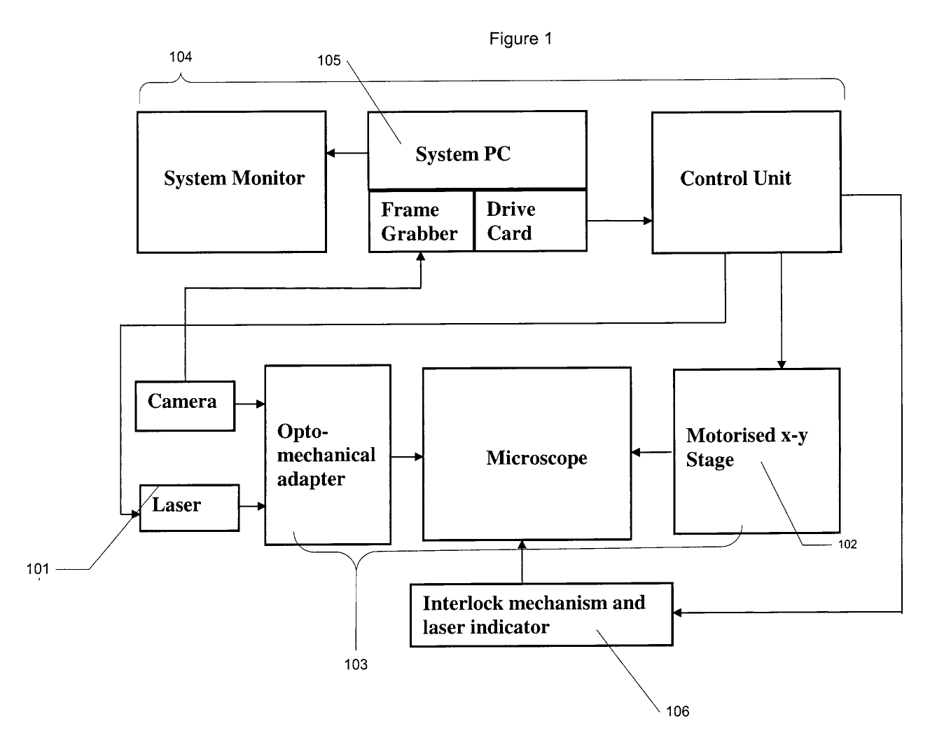

The methods of this invention comprise the use of an apparatus (exemplified in FIG. 1 in a preferred embodiment), comprising:

(a) a light source (101);

(b) a stage (102); and

(c) a light direction system (103) for directing light from the light source onto specific regions of stage. The light source is preferably focused onto a matrix placed on the stage so as to, directly or indirectly, micromanipulate biological material placed on the matrix. Such direct micromanipulation includes use of the light to dissect or otherwise separate materials, e.g., through sorting of cells or micro-discrimination of tissues. Indirect micromanipulation includes the use of the laser to modify the materials or the matrix for the materials in such a manner that the materials become differentiated, e.g., through alteration of cell growth or organization.

In a preferred embodiment, micromanipulation may be effected by ablation of the biological material or destruction of the matrix upon which the material is placed. Preferably, the apparatus additionally comprises a viewing system, for imaging the substrate, and the biological material. Also preferably, the IMM apparatus additionally comprises a controller for the system to direct light to specific regions on the substrate. IMM devices comprising components among those useful herein are described in U.S. Pat. No. 4,624,915, Schindler et al., issued Nov. 25, 1986, and U.S. Pat. No. 4,629,687, Schindler et al., issued Dec. 16, 1986, both of which are incorporated by reference herein.

Light Source:

The light source (101) comprises a source of focused light of sufficient wavelength and intensity so as to micromanipulate biologic materials on a matrix. In a preferred embodiment, the light is effective to ablate the biological material. In another embodiment, the light is effective to disrupt the matrix upon which the material is placed. (As referred to herein, “disrupt” means to substantially cut, erode, melt, weld, vaporize, or otherwise ablate substrate material.) Preferably, the light source is a laser, preferably emitting light at a wavelength of at least about (≧) 300 nanometers (nm). In particularly preferred embodiments, the laser light has a wavelength ≧600 nm. (As referred to herein, such emission wavelengths refer to the peak wavelength of emission by the light source.)

Preferably, the laser emits light at visible, near-infrared or infrared frequencies. In one embodiment, the laser is an argon laser capable of emitting light in ultraviolet and visible wavelengths between about 300 and 560 nanometers. In another embodiment, the laser is an argon-krypton laser, such as are available from a variety of commercial sources, such as supplied by Lexel, Inc., Sunnyvale, Calif., U.S.A. Preferably the light has a wavelength of at least about 600 nm, more preferably at least about 700 nm, more preferably at least about 750 nm. In one embodiment, a preferred laser is a diode laser. A particularly preferred diode laser emits light at a wavelength of about 790 nm, such as supplied by Intelite, Inc., Minden, Nev., U.S.A. In a preferred embodiment comprising a diode laser, the apparatus comprises an aspheric lens and iris to focus the laser light and reduce any ellipticity of the light beam.

Stage:

The stage (102) is a structure which supports and positions the matrix and biological material supported on the matrix in a manner that facilitates focusing of light onto the matrix. Preferably the stage functions to move the matrix in a plane that is substantially perpendicular to the direction of the light. The stage may be permanently affixed to the apparatus, removably affixed to the apparatus, or may comprise parts some of which are permanently affixed and others that are removable. (As referred to herein, a “permanently” affixed structure is one that is not removed during routine use of the apparatus. As referred to herein, “removably affixed” is the attachment of two parts in such a manner that one part is secured to and does not substantially move relative to the second part during routine use of the apparatus, but may be removed from the second part using moderate physical force. Such removably affixed attachments include clamps, clips, and other physical devices, adhesives, and electrostatic bonding.) Preferably, the stage comprises a platform comprising a matrix, wherein the platform is removably attached to a mechanism for moving the platform relative to the light. Such mechanisms are discussed further, herein.

Matrix:

In use, the apparatus comprises a matrix which serves as a substrate for biological material. The biological material is in contact with the matrix, such as by being embedded in, or otherwise in substantial contact with, the matrix so that heating of a region of the matrix during operation of the apparatus will effect heating of the biologic material in contact with that region.

In a preferred embodiment, the matrix comprises a light-absorbing material, wherein the light absorbing material selectively absorbs light in a range of wavelengths including the wavelength of light emitted by the light source. As referred to herein, a material that “selectively absorbs” light exhibits a single significant peak of absorption as a function of wavelength. In one embodiment, the light source emits light having a wavelength of from about 300 to about 560 nm, and the light absorbing material absorbs light having a wavelength of from about 300 to about 560 nm. In a preferred embodiment, the light has a wavelength of from about 750 nm to about 800 nm, and the light absorbing material absorbs light at a wavelength of from about 750 to about 800 nm. (As referred to herein, such emission and absorption wavelengths refer to the wavelength of maximum peak emission or absorption.)

Preferably, the light absorbing material comprises a light absorbing dye, pigment, or metal. Also preferably, the light absorbing material is non-toxic (i.e., does not kill or otherwise affect the biological materials, except through heating in the methods of this invention) and does not substantially absorb visible light below about 600 nm wavelength so as to significantly inhibit imaging of fluorescence emitted from fluorescent labeled biological material. In a preferred embodiment, wherein a laser emits light at near infrared or infrared wavelengths, such dyes and pigments among those useful herein include (2-[2[2-(1,1,3-trimethy-2H-benzo[e]-indol-2-ylidene)-ethylidene]-1-cyclohexen -1-yl]-ethenyl]-1,1,3-trimethy-1H-benzo[e]indolium-4-0-methylbenzylsulfonate, supplied as SDC7047, by H.W. Sands Corp., Jupiter, Fla., U.S.A. Another preferred pigment is supplied as Epolight VI-30 by Epolin, Inc., Newark, N.J., U.S.A.

Preferably, the matrix comprises a film, preferably a thermoplastic film. As referred to herein, a “film” is a sheet of material having a length and width in two dimensions that are each substantially greater than the sheet'thickness in the third dimension, having a first surface and an opposite second surface each defined by the length and width of the film. Preferred films useful herein include those comprising polyolefins, polyesters, polyvinylidene chloride, polycarbonates, and mixtures thereof. A preferred film comprises polyvinylidene chloride. Preferably the film is from about 5 microns to about 50 microns thick.

In a preferred embodiment, the biological material matrix comprises an admixture of a thermoplastic polymer and the light-absorbing material. In an alternative embodiment, the substrate comprises a thermoplastic polymer film coated with the light-absorbing material.

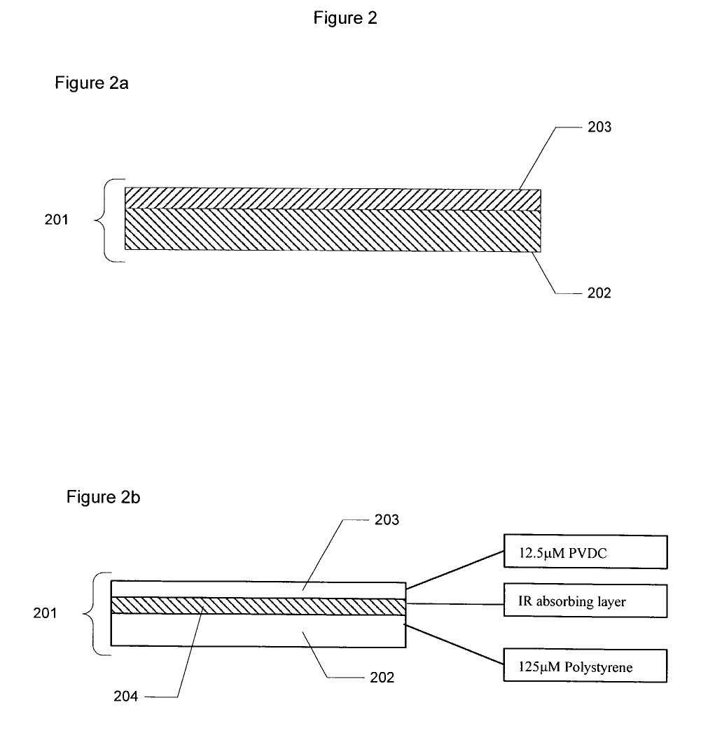

In one embodiment, the matrix (with the light absorbing material admixed or coated) is used in the methods of this invention as a single layer of material (a “mono-layer matrix.”) In another embodiment, the matrix is part of a “bi-layer matrix composite,” comprising a film, a light absorbing material, and an additional structure for supporting, holding, containing, or otherwise securing the biological material during the use of the apparatus. Preferably, as depicted in FIG. 2, the bi-layer matrix composite (201) comprises a substantially planar carrier (202), a substantially planar film (203) in substantial contact with a planar surface of said carrier, and a light absorbing material. As referred to herein, a “substantially planar” surface is, or is capable of being, flat having substantially two-dimensional geometry considering the surface as a whole, although it may have surface irregularities in a third dimension. It should be understood that bi-layer matrix composites are not limited to structure comprising two layers, but in some embodiments may comprise three or more layers, such as additional carrier materials. Also, as referred to herein, “in substantial contact with” refers to direct or indirect contact of at least one major surface the matrix with a major surface of the carrier, so that the carrier supports and is affixed to the matrix. Preferably, the carrier is removably affixed to the matrix, so that the matrix may be separated from the carrier by moderate physical force during routine use of the apparatus.

In one bi-layer matrix composite embodiment, as depicted in FIG. 2a, the light absorbing material is admixed with the material of the film layer (203). In another embodiment, light absorbing material is admixed with the material of the carrier, so as to serve as a coating on the matrix. In another embodiment, as depicted in FIG. 2b, the light absorbing material is a coating on the film, forming a layer (204) between the film (203) and the carrier (202).

Preferably, the carrier comprises a material selected from the group consisting of glass and plastic, preferably plastic, such as polycarbonate, polyester, and polystyrene. A preferred carrier comprises polyester. Preferably, the carrier is substantially transparent to the light emitted by the laser light source. Also preferably, the melting point of the carrier is equal to or higher than the melting point of the matrix.

In one embodiment, the film is affixed to the carrier with an adhesive. In another embodiment, the film is affixed to the carrier by electrostatic, friction or other means other than the use of an adhesive. Preferably, the film is laminated to the carrier, such as by coextrusion.

In a preferred embodiment, the platform comprises a rigid or semi-rigid plate support for the biological material and matrix, preferably comprising a structure conventionally used for supporting or containing biologic materials during research, or a modification thereof. Preferably, the plate comprises a substantially planar member. A planar matrix is affixed to the planar member of the plate, either as a mono-layer matrix or as a bi-layer matrix composite. It should be understood that the structure formed by affixing a mono-layer matrix to a plate forms, in some embodiments, a structure which is itself a bi-layer matrix composite, where the carrier comprises the planar member of the plate.

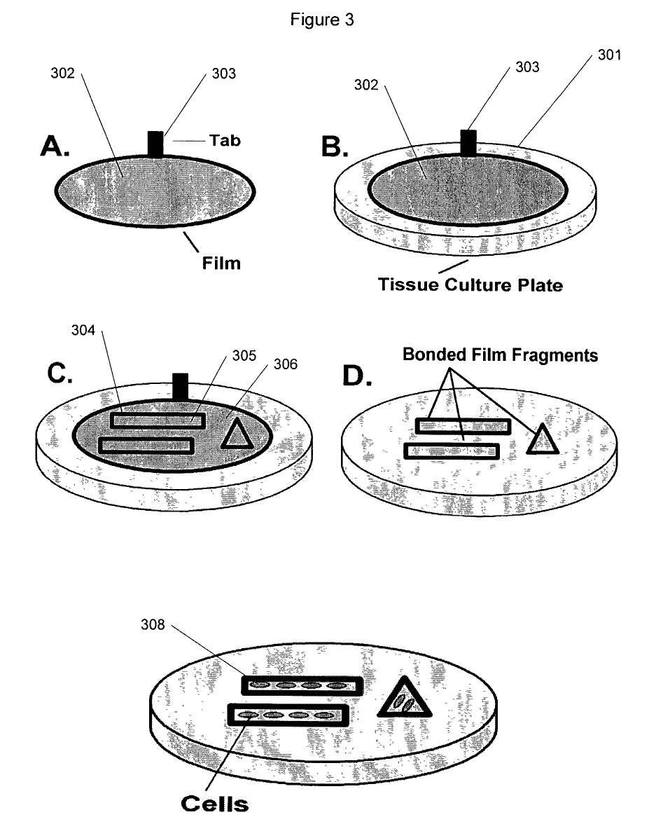

In one embodiment, the plate is a slide, such as a microscope slide, or similar flat object. In another embodiment, the plate is a container having sides and a substantially planar bottom member, such as a cell culture dish (e.g., a “Petri” dish). (As referred to herein, such directional terms as “top,” “bottom,” and “over” are used to define the spacial orientation of the parts of a structure relative to one another, and not necessarily their absolute spacial orientation relative to the overall apparatus or user.) In a preferred embodiment, exemplified in FIG. 3, the plate is a culture plate (301), upon which a matrix is applied. In a preferred embodiment, the platform comprises a 35 mm culture plate.

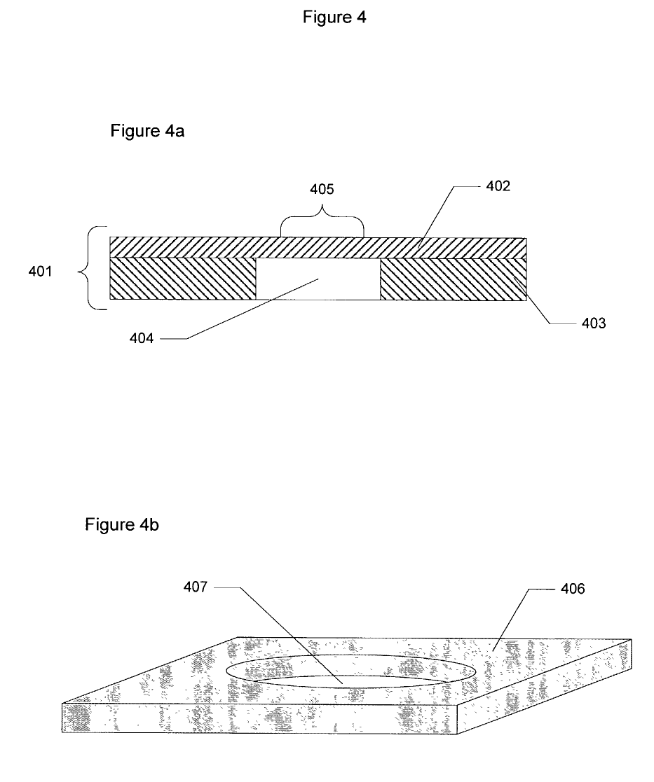

In one embodiment of this invention, exemplified in FIG. 4, the platform (401) comprises a matrix (402) and a carrier (403), wherein the carrier has one or more apertures (404), and at least a portion (405) of the matrix (402) extends over an aperture. In a preferred embodiment, the carrier is a plate. In one such embodiment, depicted in a perspective view in FIG. 4b, the plate (406) comprises a single member having one or more apertures (407). In another embodiment, the plate comprises two or more members configured so that together they define a substantially planar surface having one or more apertures. The platform is configured in such a manner so that a region of the matrix extends over at least a portion of at least one aperture in the plate. In such an embodiment, the matrix is in contact with one or more edges or other regions of the plate, such that the matrix is secured by the plate but extends over the aperture. Preferably, the matrix comprises a film that is releasably secured to the plate, so that the matrix may be readily removed from the plate during routine use of the apparatus.

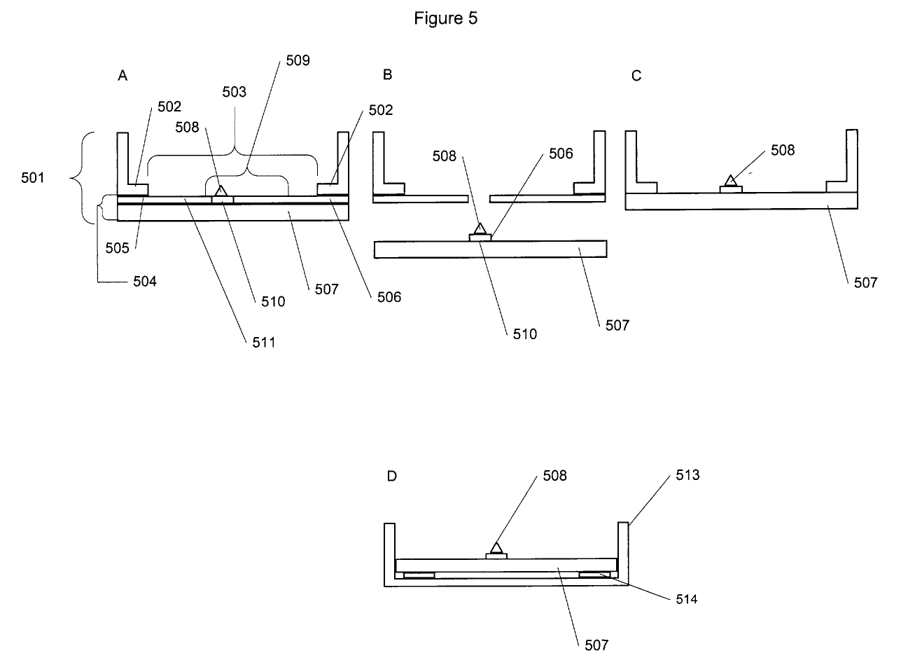

A preferred platform comprises a plate having an aperture, and bi-layer matrix composite extending over the aperture. A preferred platform (501), depicted in a cross-sectional view in FIG. 5, comprises:

(a) a substantially planar plate (502) having an aperture (503); and

(b) a matrix (504) which in substantial contact with a surface (505) of the plate (502); wherein a region of the matrix extends over the aperture. (As referred to herein, a “region” of the matrix consists of a portion of the matrix having surfaces that consist of part of the first and second surfaces of the matrix.) Such a matrix is supported by the plate, such as by being releasably secured to a surface of the plate. In reference to FIG. 4, that region (405) of the matrix (402) that is over the aperture (404) is not in direct contact with the plate (403). A preferred plate is a slide, culture dish, or other commonly used container for biological materials. In one embodiment, the matrix is a mono-layer matrix. In a preferred embodiment, the matrix is part of a bi-layer matrix composite. Preferably, the matrix of the composite is in direct contact with the plate, such that the matrix is between the plate and the carrier of the bi-layer composite.

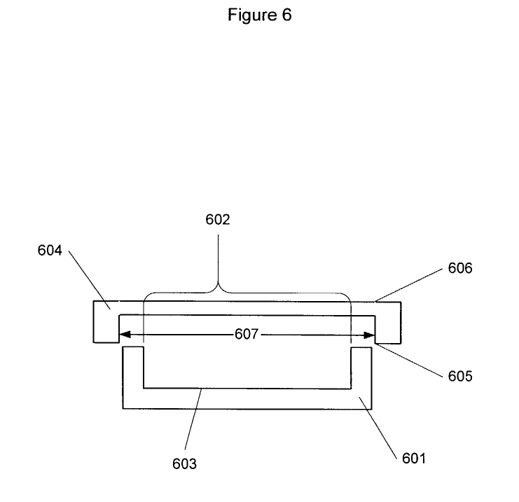

In a preferred embodiment, as depicted in a cross-sectional view in FIG. 6, a plate (601) comprises a substantially cylindrical culture dish having an open top (602) and a substantially planar bottom (603). In a preferred embodiment, the culture dish has a diameter of about 35 mm. In a preferred embodiment, the platform also comprises a substantially cylindrical lid (604) having an open end (605), a closed end (606), and a diameter (607) slightly larger than that of the dish so that the open end of the lid engages and seals the open end of the dish. Preferably, the lid releasably engages the dish, and seals the dish through a friction fit to the dish. In one embodiment, the planar bottom of the dish comprises one or more apertures, as an apertured culture dish of this invention. In another embodiment (as depicted in FIG. 6), the planar bottom is solid (i.e., does not have any apertures).

In a preferred embodiment, as depicted in FIG. 5, the platform (501) comprises a culture dish having bottom plate (502) with an aperture (503), and a bi-layer matrix composite (504) extending over the aperture, where the matrix (506) of the composite is in direct contact with the side (505) of the plate opposite the top of the dish. In such an embodiment, the matrix (506) is between the bottom of the plate (505) and the carrier (507) of the bi-layer composite.

The present invention also provides an adhesive ring for use with plates having an aperture. In one embodiment for use with circular apertures, the adhesive ring comprises a circular band of material comprising an adhesive, wherein the inner diameter of the ring is larger than the diameter of the aperture.

Preferably, the adhesive ring is substantially planar, having a first surface and an opposite second surface that are adhesive. The ring may be comprised of adhesive material, or a substrate comprising an adhesive coating. In use in one embodiment, the first surface of the adhesive ring is adhered to the plate, such that the ring is substantially centered around the aperture. A matrix of this invention is then applied to the ring, thereby being adhered to the plate while also covering the aperture. In a particularly preferred embodiment, the plate is a tissue culture plate, preferably having a lid. In a preferred embodiment, the second surface of the adhesive ring is covered with a release paper or similar covering that facilitates handling. After application of the first surface to the plate, the release paper is then removed, exposing the adhesive on the second surface.

The present invention also provides kits for use in IMM methods of this invention, comprising:

(a) a matrix comprising a light absorbing material; and

(b) an adhesive ring comprising a band of material comprising an adhesive, suitable for adhering said matrix to a support plate. In a preferred embodiment, the kit additionally comprises a substantially cylindrical culture dish having an open top and a substantially planar bottom, wherein the bottom has at least one aperture, wherein the inner diameter of the adhesive ring is larger than the diameter of the aperture.

In another embodiment, the present invention provides a biological material platform, comprising a carrier having an upper surface and a matrix in substantial contact with the upper surface, wherein at least one of the matrix and the carrier comprises a cell growth promoter. As referred to herein, a “cell growth modifier” is a material that promotes the growth or attachment of biological material. Such materials include: substrate adhesion molecules, e.g., collagen, fibronectin, and vitronection; growth factors, e.g., vascular endothelial growth factor (VEGF), fibroblast growth factor (FGF), and nerve growth factor (NGF); and mixtures thereof.

In a preferred embodiment, the carrier is a plate. In one such embodiment, the cell growth modifier is coated on the surface of a plate that is in contact with the matrix (i.e., forms a layer between the plate and the matrix). In another embodiment, the cell growth modifier is coated on the surface of the matrix that is not in contact with the upper surface of the plate. In another embodiment, the matrix comprises a thermoplastic polymer in admixture with said cell growth modifier.

In a preferred embodiment, as exemplified in FIG. 3, the matrix (302) comprises a tab (303) which is capable of aiding the mechanical separation of the matrix from the carrier/plate (301). Such a “tab” is a structure that is part of or otherwise attached to the material of the matrix which assists in removal of the matrix from the plate, such as by providing a point at which mechanical energy can be applied to the matrix.

Light Direction System:

The IMM apparatus comprises a system for directing the light onto the biological material platform in such a manner so as to effect absorption of light by the biological material or matrix. The apparatus preferably comprises a viewing system for imaging the matrix, and associated biological material. Preferably, the viewing system comprises a microscope with an objective for observing the biological material (e.g., a heterogeneous population of cells) on a platform. In one embodiment, the microscope includes a conventional binocular viewing means, support and a manual adjustment knob. Microscopes useful herein are readily available, such as from Leitz, Inc., Rockleigh, N.J., U.S.A., and Nikon, Inc., Japan. In a preferred embodiment, the apparatus comprises an inverted microscope, such as a Nikon TE2000. Preferably, the viewing system comprises a camera.

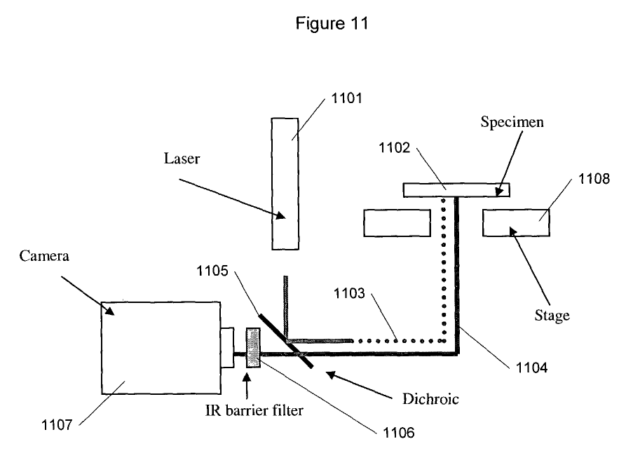

FIG. 11 depicts an apparatus embodiment comprising an optical system among those useful herein. In a preferred embodiment, a laser light source (1101) is associated with the microscope such that the beam can be focused in a path through the objective at the platform and at or around an individual cell or series of cells on the matrix (1102). Preferably, the path (1103) of laser light is coaxial with the image path (1104) of the optical system of the microscope. In a preferred embodiment, the image system comprises a dichroic mirror (1105), which is in the image path (1104) of the microscope and oriented at a 45° angle. The dichroic mirror is substantially transparent to light reflected or transmitted from the specimen, and is reflective of light from the laser light source (1101). The dichroic mirror thus effects transmission of laser light along the same optical path as the image (1104). Preferably, the system also comprises a filter (1106) which prevents any laser light from being reflected back to the camera.

The light direction system comprises a mechanism so as to focus the light on user-selected regions of the biological material or supporting matrix. Such a mechanism may comprise optical and mechanical members, such as lenses and mirrors for moving the light relative to the matrix, mechanical members for moving the matrix relative to the light, or both. In a preferred embodiment, the stage (1108) comprises a mechanism so as to move the biological material in an x and y plane perpendicular to the focused beam. Stages comprising such mechanical elements are commercially available, such as from Ludl Electronic Products, Inc., Hawthorne, N.Y., U.S.A., and Prior Scientific Instruments, Inc. Cambridge, U.K. In a preferred embodiment, the stage is capable of moving the biological material at speeds up to about 800 μ/second, with a step resolution of about 0.08 μ.

The apparatus optionally comprises a detector for distinguishing an individual cell based upon a particular optical property, or other chemical or physical property or dynamic process. The detector preferably operates by observation of the biological material through the objective. A preferred detector is a CCD.

Additional Components:

Preferably, the apparatus additionally comprises a controller (104, FIG. 1) for the system to direct light to specific regions on the platform. In a preferred apparatus, the controller comprises a laser power driver (101) and control, automatic laser shuttering control, a driver for x-y motion of the stage (102), a computer interface (105), microscope focusing control, laser controls (including safety interlocks) (106), and power supply.

Methods:

The present invention also provides methods for micromanipulating biological materials. Such methods include: ablation of unwanted materials; physical separation of materials having differing characteristics; altering the growth, organization or proportion of selected materials; and combinations thereof. In a preferred embodiment, the method is performed so that living cells in a tissue culture or tissues with a desired property are spared from a pulse of high intensity laser beam that is used to destroy unwanted materials. In this manner, a high level of cell enrichment for the defined characteristic is obtained. A significant feature of such a method is that cells cultured on slides or film may be used for enrichment. This advantage greatly increases the ultimate biological relevance of any information obtained by chemical or other analyses of the resulting sorted biological materials.

Ablation of unwanted materials is effected by irradiation of the materials directly by the laser light or, preferably, by heating a matrix in contact with the materials to a sufficient temperature so as to kill or otherwise ablate the materials. A preferred method for micromanipulating a biological material sample comprises the steps of:

(a) placing the sample on a matrix, wherein said matrix comprises a light absorbing material that selectively absorbs light at a wavelength above about 600 nm;

(b) identifying a target region of the sample and the matrix proximate to the sample; and

(c) exposing the target region to laser light having a wavelength above about 600 nm so as to heat the matrix of the region. (As referred to herein, a region of matrix “proximate to” a region of the sample is underlying in substantial contact with, or otherwise directly or indirectly adjacent to, the material so as to heat the material when the matrix is heated by light.) Accordingly, the present invention provides a biological material preparation comprising a matrix and a biological material sample in substantial contact with a planar surface of said matrix.

In one embodiment, the heat produced by illuminating the light absorbing matrix is sufficient so as to ablate or otherwise kill the biological material in the target region. In another embodiment, the heat is sufficient to destroy or otherwise produce a discontinuity in at least a portion of the matrix in the target region. In some embodiments, the heat bonds the matrix to a plate, preferably in addition to melting the matrix material at the point of illumination. In embodiments where the matrix comprises two or more component parts, the heat is preferably sufficient to destroy at least one component part. In a preferred embodiment, wherein a platform comprises a carrier (e.g., plate) and a matrix in substantial contact with the surface of the carrier, the heat is sufficient to destroy the matrix. Preferably, in such an embodiment, the carrier remains intact, and the matrix is bonded to the carrier at the target region. Also, preferably in such an embodiment, the biological material is in contact with the matrix.

In a preferred embodiment exemplified in FIG. 3, the target region (304) defines the perimeter of a first region (305). In one embodiment, the target region (304) defines a closed shape that is the boundary between the first region (305) within the perimeter of the target region (e.g., inside the closed shape), and a second region (306) that is outside the perimeter of the target region. Preferably, the heat from the light at the point of illumination of the matrix is sufficient to ablate the sample and destroy the matrix in the target region. The first region (305) is thereby separated from the second region (306), and the biological material on the first region is also separated from biological material on the second region. In another embodiment, the method comprises the additional step, after the exposing step, of excising the first region from the second region. In an alternate embodiment, the results of which are shown in FIG. 3D, the method comprises the additional step of excising the second region from the first region (305).

In an embodiment wherein the platform comprises a plate and a matrix in contact with the surface of the plate, the target, first, and second regions are on the matrix. In one embodiment, the heat is sufficient to destroy the matrix in the target region, and weld or otherwise bond the adjacent perimeters of the first and second regions to the plate. The first region can then be excised leaving the matrix and biological material in the second region intact on the plate. Alternatively, the second region is excised leaving the matrix and biological material in the first region intact on the plate. Such “excising” may be accomplished, for example, by applying physical force to the matrix so as to shear or “peel” the matrix from the plate. Preferably, the perimeters of the first and second regions are welded to the plate, so that one region remains attached to the plate when the other region is removed. In another embodiment, specific lithographic patterns may be formed on the matrix by such methods as circumscribing a region of matrix followed by excision. Depending on thickness and depth, such etched patterns can be used as barriers to cell growth or to provide information, e.g. bar codes.

A variation of the method of the present invention provides a platform comprising a thin film as a matrix mounted on a conventional tissue slide as a plate. The platform supports the cells so that the beam cuts or fuses the film to the slide around the cells for removal of unwanted cells with the film. The film is, optionally, removably affixed to the slide by lamination or with an adhesive so that the beam can cut around the wanted cells, and the unwanted cells are removed with the film from the slide. Alternatively, the beam can fuse portions of the film to the slide by beam welding. In either case, the unwanted cells and film can be stripped from the plate to leave behind discs supporting the wanted cells from film fused onto the slide. It will be appreciated that the method involving circumscribing wanted cells by cutting or fusing with the beam can be combined with the method involving killing of the unwanted cells.

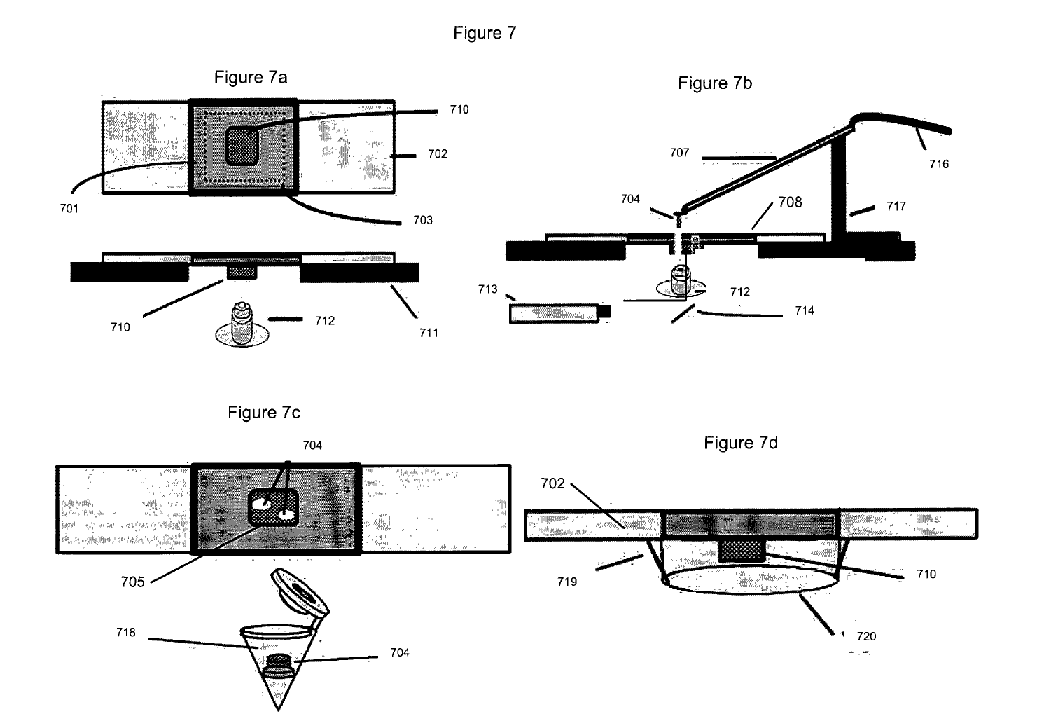

In another embodiment exemplified in FIG. 7, the present invention provides methods of micromanipulating a biological material sample, comprising the steps of:

(a) placing the sample (710) on a matrix (701) which is adhered to, or otherwise supported by, a support plate (702), wherein a region of the sample (710) and associated matrix extends over an aperture (703) in the support plate;

(b) identifying a first area (704) of the matrix within said region, and a second area (705) of the matrix contiguous to the first area;

(c) disrupting the matrix at the perimeter (706) between the first and second areas using a focused light beam, preferably generated by a laser; and

(d) excising the first area (704) and the sample on the first area.

Preferably the placing step is performed without the use of adhesives. Also preferably, the disrupting step is conducted so as to leave portions of the matrix in the perimeter intact, and the excising step is conducted by applying mechanical force to the first area so as to sever the portions. In such an embodiment, the portions of the matrix remaining intact after the destroying step are sufficient to support the first area, so that the first region does not separate from the remainder of the matrix (i.e., is not separated from the second area of the matrix) until mechanical force is applied in the excising step. In one embodiment, the mechanical force is provided by use of a device (707) that attaches (e.g., by vacuum or adhesive) to the first area on the side (708) of the matrix opposite to that of the biological material (704). After attaching to the first area, the device removes the first area (704) from the platform, as depicted in FIG. 7b.

In an alternative embodiment, depicted in FIG. 5, the method comprises:

(a) placing the sample (508) on a platform (501) comprising

(i) a plate (502) having an aperture (503) and;

(ii) a bi-layer matrix composite (504) comprising a substantially planar carrier (507), a substantially planar film (506) in substantial contact with a planar surface of said carrier, and a light absorbing material; wherein

(iii) the film (506) of said matrix composite is affixed to the bottom of the plate (505), so that a region (509) of the film is over the aperture (503) in the plate (502); and

(iv) at least a portion of the sample (508) is on said region (509);

(b) identifying a first area (510) of the matrix composite within said region, and a second area (511) of the matrix composite contiguous to the first area;

(c) disrupting the film (506) at the perimeter between the first (510) and second (511) areas using a focused light beam, preferably generated by a laser, so that the perimeter of the film of the first area is adhered to the carrier (507); and

(d) excising the first area (510) of the film and the sample (508) on the first area by removing the carrier (507) and the associated first area of the film.

The results of the excising step are depicted in Figure 5B. In a further step, depicted in Figure SC, the carrier (507) and associated sample (508) excised from the first culture apertured dish platform (501) may be adhered to the outer surface of the bottom of a second apertured culture dish (512), e.g., by use of an adhesive ring of this invention. The biological material sample may then be subjected to further research using the second culture dish. Alternatively, the carrier and associated sample may be adhered to the inner surface of the bottom of a non-apertured culture dish (513), by use of an adhesive ring (514).

In another embodiment, the present invention provides a method of micromanipulating a biological material sample, comprising the steps of:

(a) identifying a first region and a contiguous second region on a tissue growth platform, wherein said platform comprises a carrier having an upper surface and a matrix in substantial contact with said upper surface, and wherein at least one of the matrix and the carrier comprises a cell growth modifier;

(b) disrupting a perimeter area of the matrix between said first region and said second region;

(c) excising the matrix from the second region; and

(d) placing the material sample on the platform, in substantial contact with the first region, the second region, or both.

In a preferred method, the tissue sample preferentially grows on the portion of the platform that comprises the cell growth modifier. In one embodiment, the carrier comprises a cell growth promoter, so that the biological material preferentially grows on the second region. The cell growth modifier may be a coating on the carrier (i.e., between the carrier and the matrix), or admixed with the plate material. In another embodiment, the matrix comprises a cell growth promoter, so that the biological material preferentially grows on the first region. The cell growth promoter may be a coating on the matrix, or admixed with the matrix material.

The methods and apparatus of this invention my be used in a variety of laboratory techniques in a variety of biological materials. For example, in one embodiment, materials are micromanipulated by identification and removal of materials from a platform. Such methods may be used for the isolation of circumscribed pieces of tissue, although they can also be used for isolating cells or biological materials e.g. bacteria, or chromosomes. In such “film methods” a tissue (living or dead) is adhered to a matrix (e.g., film). A segment of tissue and the associated film is cut into a desired shape by the action of a laser light beam on the film. Through the intervention of a physical probe (e.g. vacuum microprobe, poker, or adhesive tip), the tissue/film segment that has been circumscribed by the illuminating source is sufficiently separated from the surrounding film so that when contacted on the surface of the film opposite to the tissue adhered side it will separate. The physical probe contacting the side of the sample that does not contain the biological material is then manipulated to pull the circumscribed tissue/film segment from its minimal connection to the surrounding film. The tissue/film segment can then be released into a tube for chemical analysis. Also, biological materials can be ablated on these surfaces.

In another embodiment, methods are used for the isolation of subpopulation of cells in tissue culture. Such methods can also be used to isolate living cells from live tissue, isolating tissue fragments from live, frozen, or fixed tissue, and separating other biological materials e.g. bacteria, chromosomes. In this “film/support method,” light absorbing films (matrices) laminated to a thermoplastic plate are placed onto the surface of a tissue culture chamber or directly laminated to the surface of a tissue culture chamber so as to provide a growth surface for living cells. These surfaces may also contain attachment factors (cell growth modifiers) to enhance the interaction between the cells and the thermoplastic materials. Cells are identified for isolation utilizing morphology or the detection of specific luminescent probes that define structure or biological activity. The cells of interest are circumscribed with an illuminating beam that is focused onto the film/support interface. The resulting dissected fragment or “cookie” consists of the desired cell(s) in association with the cut cookie that has been welded to the plate. Separation of the cookie is effected by physically peeling the film from the plate. The act of peeling separates the circumscribed cookie from the surrounding film. The cookie remains associated with the plate or the surface of the tissue culture chamber as a consequence of the physical attachment of its periphery to the plate or growth surface of the tissue culture chamber. Following the removal of the film, the remaining cookies containing the desired cells are incubated in media containing the appropriate growth or differentiation factors to support growth. This method preferably does not result in the removal or detachment of the isolated cell from the plate. In another method, cells may be isolated through the process of ablation. Unwanted cells are removed by illuminating the contact region between these cells and the film/support. The heat generated by illuminating the light absorbing material in this localized region results in the production of sufficient heat to kill and ablate biological materials. Following ablation, the disrupted material is removed from the culture through a change in media. This method can be utilized to isolate subcellular fragments such as chromosomal segments.

Methods of this invention may also be used for tissue micro-dissection. Such methods involve the identification, separation, and isolation of tissue fragments of interest for chemical and genetic analysis. The tissue may be living (an explant), frozen (cryotome) or fixed (formalin). Tissue micro-dissection may be performed using either the film or film/support methods described above. Such methods can be used with thick tissues, e.g. including samples about 200 microns in thickness.

Isolation of live cells from tissue explants or biopsies can also be performed in other embodiments. Tissues may be sectioned to a desired thickness with a vibratome. Using either the film or film/support method, micro-regions of tissue containing cells are isolated from the tissue mass. Growth media and attachment factors are supplied to promote cell growth. Alternatively, live tissue can be disaggregated through the use of enzymes and chelators. The resulting disaggregated cells can be seeded onto a film/support surface. Following adherence, these cells can be sorted as described for the film/support method. Non-adherent cells (e.g. lymphoid) may be sorted in a similar fashion. Ablation methods may also be employed as a secondary means of enriching for desired cells.

Frozen and fixed tissues may also be used in methods of this invention. In such methods, sections of frozen tissue are cut by a microtome in a cryostat. The cut tissue falls onto a film or film/plate surface. This substrate is then processed as described above for a film or film/plate system. Ablation may also be used as a secondary means to remove unwanted material.

In cell sorting embodiments, cells derived from cultures or tissue explants are seeded on the matrix, using either film or film/support methods. Selected cells are then processed as described. The film/plate method provides a means to maintain sterility and not interfere with the attachment of anchorage dependent cells. Multiple rounds of attachment and detachment can affect cell viability, cloning ability, and differentiated states. The method may also be employed to separate lymphoid cells, bacteria and other cells that do not form attachment factor mediated association with the film. Such cells may have sufficient adherence to the film or can be artificially anchored by the addition of adhesion molecules to the film surfaces. Adhesion materials useful herein include polyphenolic proteins, such as those sold as Cell-Tak™by BD Biosciences, Lexington, Ky., U.S.A.

The IMM methods of this invention can also be employed for isolating chromosomes and subcellular organelles, e.g. nuclei. Biological material is placed on a film or film/plate. Adherence to the films can be enhanced by application of adherence factors (e.g. Cell-Tak). Utilizing appropriate identification methods (e.g. chromosome spreading, luminescent nucleotide probes, morphology, luminescent organelle specific probes), these structures can be isolated through the use of the methods of this invention. Also, regions of chromosomes may be made available for amplication (polymerase chain reaction) through the ablation of undesirable regions.

Utilizing the techniques of ablation and cookie cutting, it is also possible to modify growing cell populations to maintain specific ratios between cell types, introduce specific geometries between cell types and, in the case of primary cell cultures, remove or “weed” undesirable cells (usually fibroblasts). In one embodiment, cells grown on light absorbing films or film/plate in a tissue culture chamber are monitored over time. Utilizing morphology or luminescent probes capable of discriminating between cell types, ablation of cells can be performed in a systematic way to introduce or maintain specific cell type to cell type ratios. In another method, by daily monitoring the cells in culture, the ablation methods described above may be employed to remove undesirable cells from the primary culture (“weeding”). Ablated cells detach from the growth surface and are eliminated through the removal and replacement of media. In another embodiment, specific geometries can be created that provide a bounded region for cell growth into uniquely shaped cell populations. Utilizing a “cookie cutting” method, specific geometric areas are cut into the matrix. These surfaces are seeded with a unique cell type. As growth continues, the film is removed leaving the welded cookie with associated cells remaining welded to the tissue culture surface. This plate is then seeded with another cell type. Cells associated with the attached cookie will proliferate to the boundary of the cookie (depending on the width and the thickness of the etched boundary, cells will either migrate across slowly or not at all), while the other cell types populate the remainder of the plate. In this manner, microdomains with a unique user defined geometry containing one cell type can be created within a population composed of other cell types. In one embodiment, this method is useful for creating “artificial” distributions of microtumors within normal cell populations in tissue culture. This method may be enhanced by the introduction of layers of light absorbing materials (substrates) whose surfaces are covered with attachment factors that are specific for different cell types. In this manner multiple populations of geometric specific cell growth areas may be engineered into the tissue culture environment.

In another method, by controlling the motion of the laser on the substrate, desired patterns can be introduced into the material for identification and experimentation. By controlling the intensity of the beam, etched regions can be prepared that inhibit or decrease cell migration across them. In this manner, one can provide a variety of patterned boundaries to control the orientation and spatial pattern of cell growth. Additionally, photolithographic means may be used to etch identifying codes or write experimental details on the films.

The following are non-limiting Examples of the apparatus, materials, and methods of this invention.

### EXAMPLE 1

A biological material platform according to this invention is made as follows. A thermoplastic film comprised of composed of SARAN®(polyvinylidene chloride, Dow Chemical, Midland, Mich., U.S.A.) is solvent cast containing 4 gms of SARAN and 0.08 gms Epolight VI-30 (Epolin, Inc., Newark, N.J., U.S.A.) which has an absorption maximum at λ=790 nm. Tetrahydrofuran is used as the solvent. The solution is left to evaporate and the resulting film is left to dry and then adhered as a matrix to a tissue culture plate as a plate, with the dye surface in contact with the polystyrene surface of the tissue culture plate. Adherence of the film to the plate is produced by the uniform application of pressure.

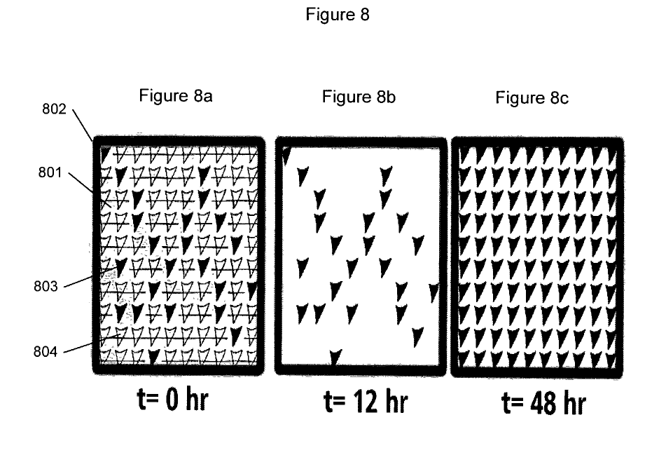

As exemplified in FIG. 8, a cell culture (801), in an appropriate growth media, is then placed on the film in the culture plate (802). The plate is placed in an IMM apparatus, and an infrared laser beam is scanned across the cell culture to avoid selected cells (e.g., 803), and ablate undesired cells (e.g., 804). After about twelve hours, the cells not exposed to ablation maintain their attachment to the film, and are viable (FIG. 8b). After about forty eight hours, the selected cells have divided and have re-populated the growth surface (FIG. 8c). This method lends itself to both positive and negative selection.

### EXAMPLE 2

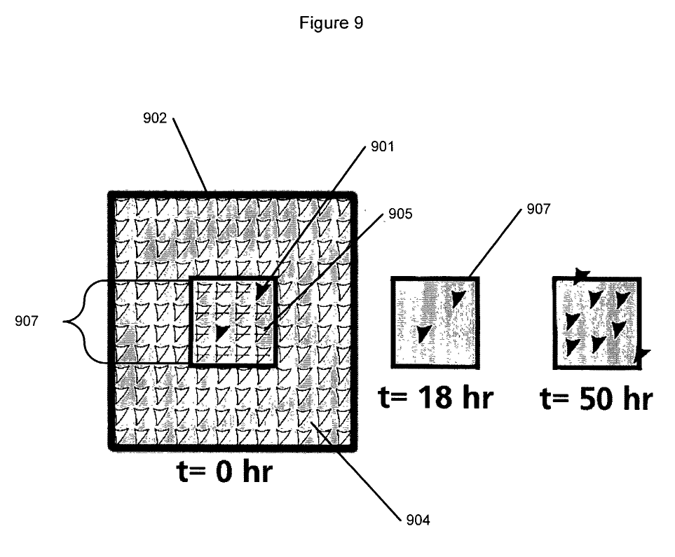

A platform made as in Example 1 (exemplified in FIG. 9) is used in another method of this invention. A cell culture is added to the plate (902) of Example 1. Cells (e.g., 901, 905) adhering to the film (904) are identified and are circumscribed by an infrared laser beam. The heat generated by the absorption of the focused laser beam by the light absorbing material in the film causes the film to melt and fuses the circumscribed film (a “cookie”, 907) to the underlying tissue culture plate. Laser ablation of individual cells is then performed to destroy unwanted cells (e.g., 905) within the cookie. The film containing the unselected cells (901) is then removed by manually peeling it off the tissue culture plate.

For isolating more than one cell or subpopulation of cells, this process is repeated in individual wells of multi-well tissue culture plates each lined with a film comprising a light absorbing material. These cells are destroyed or further cultured in a separate dish. The selected cells, adhering to the isolated cookie on the tissue culture plate, are now ready for clonal expansion or differentiation.

### EXAMPLE 3

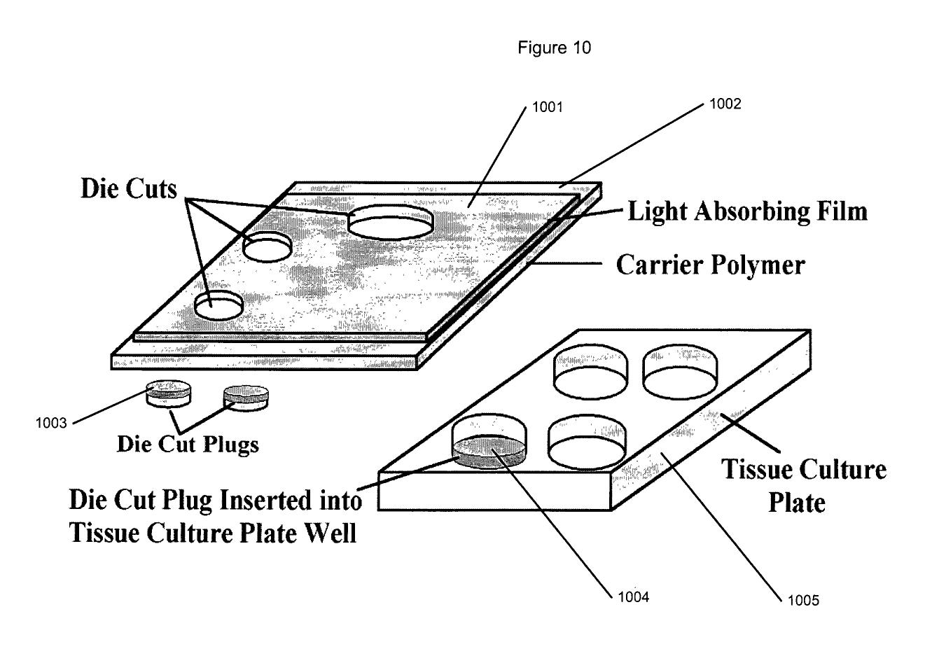

As exemplified in FIG. 10, a light absorbing film (1001) is either laminated to a carrier plastic (1002) (e.g. polystyrene), or co-extruded with a carrier plastic, resulting in two adhering sheets of plastic as shown in the figure. Sheets of the film and carrier are prepared and cut to size. The sheets may be made sterile by, e.g., UV illumination, and then die cut to form die cut plugs of film/carrier (1003) to be used as inserts for culture wells (1004) in a tissue culture plate (1005).

### EXAMPLE 4

As exemplified in FIG. 7, a biological material platform according to the present invention is made as follows. A plastic microscope slide (702) with a central aperture (703, dotted lines define opening) is used as a plate, over which a thin (2 to 10 micron) film (701) of a polymer, such as polyvinylidene chloride, is evenly and tautly attached, as the matrix. The film is made more absorptive to visible laser light by application of a light absorbing material, e.g., an inert darkening agent, such as carbon black. A tissue slice (710), prepared by one of various common methods, is placed on the darkened film and mounted on the movable stage (711) of an inverted microscope (e.g., Nikon Diaphot 300) with the tissue side (710) facing the objective (712). Visual examination by one of any effective optical means (phase, transmission, interference, fluorescence, etc.) is conducted either by direct observation through the eyepieces, or by images captured by a digital camera or other electronic image capture device and viewed on a monitor, and the desired cell(s) identified. As shown in FIG. 7b, a coaxial beam from a solid-state laser (713) (e.g., Power Technology, Model ACMT 60/ 3525), is directed by a dichroic mirror (714) through the objective (712) resulting in a focused beam of light with radius between 1-5 microns. The laser beam is then used to circumscribe the desired cell(s) (704), either by scanning the beam through the intervention of beam positioning mirrors or through the movement of the stage, simultaneously cutting both the tissue and the adhering film as the first step of the isolation process. Once the supporting film is severed the circumscribed region (cookie) (704) is maintained in position by attachment through thin plastic threads resulting from the melting process. The circumscribed cookie (704), consisting of the plastic disk cut from the original support and the selected and now isolated cell(s), is removed by use of a vacuum that is applied through the tip of a micropipette (707) that is connected through a tube (716) to a vacuum pump. The vacuum micropipette is anchored to the stage by attachment to a movable (z-direction) holder (717). As depicted in FIG. 7c, following the micro-dissection in which cell(s) have been removed from the surrounding tissue (gaps in tissue resulting from micro-dissection), the isolated cell(s)/film complex (704) is placed into an analytical tube (718) or other suitable micro container for subsequent processing and genomic or proteomic analysis. In micro-dissection approaches that utilize fresh tissue, the tissue holder (702) is modified as shown in FIG. 7d to support a covering that can maintain buffer or media throughout the procedure. In this slide, the tissue and film are situated with a cylindrical well that has sloping sides (719). A cap with an optically transparent bottom (720) forms a liquid tight seal by means of a pressure fit with the walls of the well. In this way, the sample (710) can be continuously exposed to media or buffer which is contained in the capped well during micro-dissection and the isolated sample may be removed using the vacuum micropipette probe as described above.

### EXAMPLE 5

A method of micromanipulating according to this invention comprises the use of a film according to this invention, comprising an infrared light absorbing material and a growth factor as a cell growth modifying material. As exemplified in FIG. 3, the film (302) is pressed tightly or laminated onto a tissue culture surface plate (301). The tissue culture plate with the adhering film is placed on the stage of a microscope. A beam of laser light from an infrared diode laser is focused onto the surface of the film. The absorption of light by the dye at the incident point on the film is converted into heat. Through the use of galvanometric mirrors or a computer controlled two dimensional stage, the beam of light or the film, respectively, is moved to produce a desired pattern or shape (e.g., 305) on the surface of the film. The heat produced at the incident point melts the film and the resulting shape or pattern is bonded or welded to the plate along the melted edges (304) of the cut film fragment. When the film is manually peeled away from the surface, the cut fragments and shapes (e.g., 305) are left associated with the tissue culture surface, as depicted in FIG. 3d. Cells (308) seeded onto the tissue culture plate will only grow on the shapes or patterns formed by the film surface. The cells divide to fill the space comprised by the pattern or shaped fragment.

In another embodiment, this method is modified to utilize the film as a negative mask with no growth modifying surfaces, or to provide another type of adhesion surface in conjunction with the tissue culture surface. In this way two different growth promoting surfaces may be manufactured with specific topological relationships to each other within a tissue culture plate. This can be in the form of patterned cell growth or the formation of cellular islands e.g. regions resembling microtumors. This method is also be extended to provide more than two diverse types of growth or inhibitory regions within a cell culture system. In this embodiment of the method, the sequential and additive adherence, cutting/welding of films containing the desired growth or inhibitory material can result in the production of multiple regions of growth and inhibition demonstrating specific topological relationships to each other.

The examples and other embodiments described herein are exemplary and not intended to be limiting in describing the full scope of compositions and methods of this invention. Equivalent changes, modifications and, variations of specific embodiments, materials, compositions and methods may be made within the scope of the present invention, with substantially similar results.

What is claimed is:

1. A micromanipulating apparatus, comprising:
(a) a laser light source emitting light at a wavelength of at least about 600 nm;
(b) a material matrix, comprising a light-absorbing material, wherein said light absorbing material selectively absorbs light in a range of wavelengths including the wavelength of said light; and
(c) a light direction system for directing light from said laser light source onto said matrix,
wherein said light absorbing material converts said light to heat so as to effect melting of said matrix at the point where said light contacts said absorbing material.

2. A micromanipulating apparatus, according to claim 1, additionally comprising a viewing system, for imaging said matrix.

3. A micromanipulating apparatus, according to claim 2, additionally comprising a controller that controls said light direction system so as to direct said light to specific regions on said matrix.

4. A micromanipulating apparatus, according to claim 1, wherein said light is infrared or near-infrared.

5. A micromanipulating apparatus, according to claim 4, wherein said light absorbing material comprises a light absorbing dye or pigment that absorbs light at a wavelength of from about 750 to about 800 nm.

6. A micromanipulating apparatus, according to claim 4, wherein said dye or pigment is (2-[2[2-(1,1,3-trimethy-2H-benzo[e]-indol-2-ylidene)-ethylidene]-1-cyclohexen-1-yl]-ethenyl]-1,1,3-trimethy-1H-benzo[e]indolium-4-0-methylbenzylsulfonate.

7. A micromanipulating apparatus, according to claim 1, wherein said matrix comprises a substrate material selected from the group consisting of glass and plastic.

8. A micromanipulating apparatus, according to claim 7, wherein said material is plastic.

9. A micromanipulating apparatus, according to claim 8, wherein said matrix is polyvinylidene chloride film.

10. A micromanipulating apparatus, according to claim 8, wherein said matrix comprises an admixture of said substrate material and said light-absorbing material.

11. A micromanipulating apparatus, according to claim 8, wherein plastic is coated with said light-absorbing material.

12. A micromanipulating apparatus, according to claim 1, wherein said apparatus comprises a bi-layer matrix composite comprising said matrix, wherein said matrix is substantially planar and is in substantial contact with a planar surface of a substantially planar carrier.

13. A micromanipulating apparatus, according to claim 12, wherein said matrix comprises a thermoplastic film.

14. A micromanipulating apparatus, according to claim 13, wherein said light absorbing material is coated on said matrix.

15. A micromanipulating apparatus, according to claim 13, wherein said thermoplastic film comprises polyvinylidene chloride.

16. A micromanipulating apparatus, according to claim 12, wherein said carrier comprises glass or plastic.

17. A micromanipulating apparatus, according to claim 16, wherein said carrier comprises a plastic selected from the group consisting of polycarbonate, polyester, and polystyrene, and mixtures thereof.

18. A micromanipulating apparatus, according to claim 1, wherein said apparatus comprises a platform comprising said matrix and a substantially planar support plate, wherein said matrix is substantially planar and is in substantial contact with a planar surface of said plate.

19. A micromanipulating apparatus, according to claim 18, wherein said matrix comprises a thermoplastic film.

20. A micromanipulating apparatus, according to claim 19, wherein said thermoplastic film comprises polyvinylidene chloride.

21. A micromanipulating apparatus, according to claim 18, wherein said light absorbing material is coated on said matrix.

22. A micromanipulating apparatus, according to claim 18, wherein said matrix is in substantial contact with a planar surface of a substantially planar carrier.

23. A micromanipulating apparatus, according to claim 22, wherein said matrix is affixed to said planar surface of said plate.

24. A micromanipulating apparatus, according to claim 22, wherein said carrier is affixed to said planar surface of said plate.

25. A biological material platform for use in micromanipulating, comprising a substantially planar matrix and a light absorbing material that selectively absorbs light at a wavelength of at least about 600 nm.

26. A biological material platform according to claim 25, wherein said light absorbing material absorbs light at a wavelength of from about 750 nm to about 800 nm.

27. A biological material platform, according to claim 26, wherein said absorbing material comprises (2-[2[2-(1,1,3-trimethy-2H-benzo[e]-indol-2-ylidene)-ethylidene]-1-cyclohexen-1-yl]-ethenyl]-1,1,3-trimethy-1H-benzo[e]indolium-4-0-methylbenzylsulfonate.

28. A biological material platform, according to claim 25, wherein said matrix is a film in substantial contact with a planar surface of a substantially planar support carrier.

29. A biological material platform, according to claim 28, wherein said matrix comprises a thermoplastic polymer.

30. A biological material platform, according to claim 29, wherein said polymer is selected from the group consisting of polyester, polyvinylidene chloride, polycarbonate, and mixtures thereof.

31. A biological material platform, according to claim 30, wherein said polymer comprises polyvinylidene chloride.

32. A biological material platform, according to claim 28, wherein said carrier comprises a plastic selected from the group consisting of polycarbonate, polyester, and polystyrene, and mixtures thereof.

33. A biological material platform, according to claim 32, wherein said carrier comprises polystyrene.

34. A biological material platform, according to claim 25, wherein said matrix is in substantial contact with a planar surface of a support plate.

35. A biological material platform, according to claim 34, wherein said matrix comprises a thermoplastic polymer.

36. A biological material platform, according to claim 35, wherein said polymer is selected from the group consisting of polyester, polyvinylidene chloride, polycarbonate, and mixtures thereof.

37. A biological material platform, according to claim 36, wherein said polymer comprises polyvinylidene chloride.

38. A biological material platform, according to claim 34, wherein said plate is a microscope slide or tissue culture plate.

39. A biological material culture platform according to claim 37, wherein said matrix is substantially planar and comprises a tab which is capable of aiding the mechanical separation of said matrix from said plate.

40. A biological material preparation comprising a tissue platform of claim 25 and a biological material sample in substantial contact with a planar surface of said matrix.

41. A biological material preparation according to claim 40, wherein said biological material sample comprises a cell culture.

42. A biological material preparation according to claim 40, wherein said biological material sample comprises a tissue specimen.

43. A method of micromanipulating a biological material sample, comprising the steps of:
(a) placing said sample on a matrix comprising a light absorbing material that selectively absorbs light at a wavelength of at least about 600 nm;
(b) identifying a target region of said sample and matrix proximate to said sample; and
(c) exposing said target region to laser light having a wavelength of at least about 600 nm so as to heat said matrix of said target region.

44. A method of micromanipulating a biological material sample, according to claim 43, wherein said heat is sufficient to kill said sample in said target region.

45. A method of micromanipulating a biological material sample, according to claim 43, wherein said heat is sufficient to destroy at least a portion of said matrix in said first region.

46. A method of micromanipulating a biological material sample, according to claim 45, wherein said target region defines the perimeter between a first region and a second region.

47. A method of micromanipulating a tissue sample, according to claim 46, wherein said heat is sufficient to ablate the sample and destroy the substrate in said target region.

48. A method of micromanipulating a biological material sample, according to claim 47, further comprising, after said exposing step, the step of excising said second region from said first region.

49. A platform for micromanipulating biological material, comprising
(a) a substantially planar plate having an aperture; and
(b) a matrix comprising a light absorbing material, wherein
(c) said matrix in substantially planar and is in substantial contact with a surface of said substrate; and
(d) a region of said matrix extends over said aperture.

50. A platform according to claim 49, wherein said light absorbing material absorbs light at a wavelength of from about 750 nm to about 800 nm.

51. A biological material platform, according to claim 50, wherein said absorbing material comprises (2-[2[2-(1,1,3-trimethy-2H-benzo[e]-indol-2-ylidene)-ethylidene]-1-cyclohexen-1-yl]-ethenyl]-1,1,3-trimethy-1H-benzo[e]indolium-4-0-methylbenzylsulfonate.

52. A biological material platform, according to claim 49, wherein said matrix is a film in substantial contact with a planar surface of a substantially planar support carrier.

53. A biological material platform, according to claim 49, wherein said matrix comprises a thermoplastic polymer.

54. A biological material platform, according to claim 53, wherein said polymer is selected from the group consisting of polyester, polyvinylidene chloride, polycarbonate, and mixtures thereof.

55. A biological material platform, according to claim 54, wherein said polymer comprises polyvinylidene chloride.

56. A biological material platform, according to claim 49, wherein said plate is a microscope slide or tissue culture plate.

57. A micromanipulation apparatus, comprising:
(a) a laser light source;
(b) a platform according to claim 49; and
(c) an optical system for directing light from said laser light source onto said region of the substrate;
wherein a region of said matrix is over said aperture.

58. A micromanipulation apparatus according to claim 57, wherein said laser light source emits light at a wavelength of at least about 600 nm.

59. A method of micromanipulating a biological material sample, comprising the steps of:
(a) placing said sample on a matrix comprising a light absorbing material, wherein said matrix is in substantial contact with a support plate having an aperture, and wherein a region of said matrix is over said aperture and at least a portion of said sample is placed on said region;
(b) identifying a first area of said matrix within said region, and a second area of said matrix contiguous with said first area;
(c) disrupting a perimeter area of the substrate between the first and second areas using a laser; and
(d) excising said first area and the sample on said first area.

60. A method according to claim 59, wherein said disrupting step is conducted so as to leave portions of said film in said perimeter intact, and said excising step is conducted by applying mechanical force to said first region so as to sever said portions.

61. A method according to claim 59, wherein said placing step is performed without the use of adhesive materials.

62. A method according to claim 59, wherein said laser emits light at a wavelength of at least about 600 nm.

63. A method according to claim 59, wherein said light absorbing material absorbs light at a wavelength of from about 750 nm to about 800 nm.

64. A method, according to claim 63, wherein said absorbing material comprises (2-[2[2-(1,1,3-trimethy-2H-benzo[e]-indol-2-ylidene)-ethylidene]-1-cyclohexen-1-yl]-ethenyl]-1,1,3-trimethy-1H-benzo[e]indolium-4-0-methylbenzylsulfonate.

65. A method according to claim 59, wherein said matrix comprises a thermoplastic polymer.

66. A method according to claim 59, wherein said polymer comprises polyvinylidene chloride.

67. A method according to claim 59, wherein said plate is a microscope slide or tissue culture plate.

68. A method of micromanipulating a biological material sample, comprising the steps of:
(a) placing the sample on a platform comprising
(i) a plate having an aperture); and
(ii) a bi-layer matrix composite comprising a substantially planar carrier a substantially planar film in substantial contact with a planar surface of said carrier, and a light absorbing material; wherein
(iii) the film of said matrix composite is affixed to the bottom of the plate, so that a region of the film over the aperture in the plate; and
(iv) at least a portion of the sample is on said region;
(b) identifying a first area of the matrix composite within said region, and a second area of the matrix composite contiguous to the first area;
(c) disrupting the film at the perimeter between the first and second areas using a focused light beam, preferably generated by a laser, so that the perimeter of the film of the first area is adhered to the carrier; and
(d) excising the first area of the film and the sample on the first area by removing the carrier and the associated first area of the film.

69. A method according to claim 68, wherein said light absorbing material absorbs light at a wavelength of from about 750 nm to about 800 nm.

70. A method according to claim 69, wherein said absorbing material comprises (2-[2[2-(1,1,3-trimethy-2H-benzo[e]-indol-2-ylidene)-ethylidene]-1-cyclohexen-1-yl]-ethenyl]-1,1,3-trimethy-1H-benzo[e]indolium-4-0-methylbenzylsulfonate.

71. A method according to claim 68, wherein said matrix comprises a thermoplastic polymer.

72. A method according to claim 68, wherein said polymer comprises polyvinylidene chloride.

73. A method according to claim 68, wherein said plate is a microscope slide or tissue culture plate.

74. A biological material support, comprising a support plate having an upper surface and a matrix in substantial contact with said upper surface, wherein at least one of said plate and said matrix comprises a cell growth modifier.

75. A biological material support according to claim 74, wherein said cell growth modifier is coated on said upper surface of the plate.

76. A biological material support according to claim 74, wherein said cell growth modifier is coated on the surface of said matrix that is not in contact with said upper surface of the plate.

77. A biological material support according to claim 74, wherein said matrix comprises a thermoplastic polymer and a light absorbing material.

78. A biological material support according to claim 77, wherein said film comprises said thermoplastic polymer in admixture with said cell growth modifier.

79. A biological material support according to claim 74, wherein said cell growth modifier is selected from the group consisting of substrate adhesion molecules, growth factors, and mixtures thereof.

80. A biological material support according to claim 79, wherein said cell growth modifier is selected from the group consisting of collagen, fibronectin, and vitronection; vascular endothelial growth factor, fibroblast growth factor, and nerve growth factor; and mixtures thereof.

81. A method of micromanipulating a biological material sample, comprising the steps of:
(a) identifying a first region and a contiguous second region on a tissue growth platform, wherein said platform comprises a support plate having an upper surface and a matrix in substantial contact with said upper surface, and wherein at least one of said plate and said matrix comprises a cell growth modifier;
(b) disrupting a perimeter area of said matrix between said first region and said second region using a laser;
(c) excising said matrix from said second region; and
(d) placing said tissue sample on said platform, in substantial contact with said first region, said second region or both.

82. A method according to claim 81, wherein said laser emits light at wavelength of at least about 600 nm.

83. A method according to claim 82, wherein said light absorbing material absorbs light at a wavelength of from about 750 nm to about 800 nm.

84. A method according to claim 83, wherein said absorbing material comprises (2-[2[2-(1,1,3-trimethy-2H-benzo[e]-indol-2-ylidene)-ethylidene]-1-cyclohexen-1-yl]-ethenyl]-1,1,3-trimethy-1H-benzo[e]indolium-4-0-methylbenzylsulfonate.

85. A method according to claim 81, wherein said matrix comprises a thermoplastic polymer.

86. A method according to claim 85, wherein said polymer comprises polyvinylidene chloride.

87. A method according to claim 81, wherein said plate is a microscope slide or tissue culture plate.

88. A method according to claim 81, wherein said cell growth modifier is selected from the group consisting of substrate adhesion molecules, growth factors, and mixtures thereof.

89. A method according to claim 88, wherein said cell growth modifier is selected from the group consisting of collagen, fibronectin, and vitronection; vascular endothelial growth factor, fibroblast growth factor, and nerve growth factor; and mixtures thereof.
